(19) United States
(12) Patent Application Publication
(10) Pub. No.: US 2007/0184132 A1
(43) Pub. Date: Aug. 9, 2007

(54) Methods of treating canine osteosarcoma

(75) Inventor: Najla Guthrie, London (CA)

Correspondence Address: DAVIDSON, DAVIDSON & KAPPEL, LLC, 485 SEVENTH AVENUE, 14TH FLOOR, NEW YORK, NY, 10018, US

(73) Assignee: KGK Synergize Inc

(21) Appl. No.: 11/704,437

(22) Filed: Feb. 9, 2007

Related U.S. Application Data
(60) Provisional application No. 60/771,752, filed on Feb. 9, 2006.

(51) Int. Cl. A61K 36/752; A61K 31/7048; A61K 31/353; A61K 31/192; A01N 31/08

(52) U.S. Cl. 424/736; 514/27; 514/456; 514/731; 514/568

(57) ABSTRACT

Disclosed are methods and formulations for the treatment of canine osteosarcoma.

This application claim priority to U.S. Provisional Application No. 60/771,752, filed Feb. 9, 2006, which is hereby incorporated by reference in its entirety.

### BACKGROUND OF THE INVENTION

Cancer is a disease of inappropriate tissue accumulation. Chemotherapeutic agents share one characteristic: they are usually more effective in killing or damaging malignant cells than normal cells. However the fact that they do harm normal cells indicates their potential for toxicity. Animal tumor investigations and human clinical trials have shown that drug combinations produce higher rates of objective response and longer survival than single agents. Combination drug therapy is therefore, the basis for most chemotherapy employed at present (DeVita, V. T. et al., 1995, Cancer 35:98).

In dogs, osteosarcoma is the most frequently diagnosed primary bone tumor (Priester and Mc Kay, 1980). Osteosarcoma is a highly malignant tumor and pulmonary metastases occur early in the disease course (Spodnick et al., 1992). Successful chemotherapy depends on inducing selective death of tumor cells whilst sparing normal cells.

Cancer treatment requires inhibitions of a variety of factors including tumor cell proliferation, metastatic dissemination of cancer cells to other parts of the body, invasion, tumor-induced neovascularization, and enhancement of host immunological responses and cytotoxicity. Conventional cancer chemotherapeutic agents have often been selected on the basis of their cytotoxicity to tumor cells. However, some anticancer agents have adverse effects on the patients immune system.

Meloxicam was introduced as an anti-inflammatory drug before the discovery of COX-2. During recent years, experimental, epidemiological, and clinical studies have identified COX-inhibitors as promising compounds in anticancer therapy. The prognosis of osteosarcoma after surgery is poor, whilst supplementary, expensive chemotherapeutics essentially prolong survival rates by controlling growth of distant metastases. However, as stated above, their use is limited due to severe toxicity. Therefore, the ultimate goal remains to discover effective therapeutics that are affordable and which have minimal toxicity.

### OBJECTS AND SUMMARY OF THE INVENTION

It is an object of the present invention to provide a method for treating canine osteosarcoma by the administration of a therapeutically effective amount of natural plant based products to a canine in need of such treatment.

It is a further object of certain embodiments of the present invention to provide a viable alternative therapy for the treatment of canine osteosarcoma than currently available therapies.

It is a further object of certain embodiments of the present invention to provide a formulation suitable for administration to a canine and which is therapeutically effective in the treatment of osteosarcoma.

In accordance with the above objects and others, the present invention is directed in part to a method of treating canine osteosarcoma comprising administering a composition comprising an effective amount of natural plant based products to treat osteosarcoma to a canine in need of such treatment.

In certain embodiments, the present invention is further directed to a method of treating canine osteosarcoma comprising administering a composition comprising an effective amount of natural plant based products to inhibit the growth of canine osteosarcoma D-17 cells to a canine in need of such treatment.

In certain embodiments, the present invention is further directed to a composition comprising an effective amount of natural plant based products to treat osteosarcoma in a canine.

In certain embodiments, the present invention is further directed to composition comprising an effective amount of natural plant based products to inhibit the growth of D-17 cells to a canine in need of osteosarcoma cancer treatment.

In certain preferred embodiments, the present invention is further directed to a formulation suitable for the treatment of osteosarcoma in canines, the formulation comprising an effective amount of at least one of the plant based products selected from the group consisting of polymethoxylated flavones (PMF), canola phenolic acid (CPA), orange peel extract (O-PMF), limonoids, or combination thereof.

In certain embodiments, the effective amount of natural plant based products for use in accordance with the present invention to treat canine osteosarcoma is determined by the inhibition of the growth of canine osteosarcoma D-17 cells in vitro.

In certain preferred embodiments, the formulations of the present invention provide an IC₅₀ of the natural plant based products suitable for the treatment of canine osteosarcoma upon administration to a canine.

In certain preferred embodiments, the methods of the present invention are further directed to administering a formulation of the present invention to a canine in need of osteosarcoma therapy, wherein the formulation provides an IC₅₀ of natural plant based products suitable for the treatment of canine osteosarcoma.

In certain preferred embodiments, the natural plant based products for use in accordance with the present invention are selected from the group consisting of polymethoxyflavones, orange peel extracts, liminoids, canola phenolic acids, and mixtures thereof.

In certain preferred embodiments of the present invention, the natural plant based products for inclusion in the formulation of the present invention are orange peel extracts and liminoids in a 1:1 ratio.

In certain embodiments of the present invention, the formulation of the present invention further includes one or more tocotrienols.

In certain embodiments of the present invention wherein the formulation comprises one or more tocotrienols and polymethoxyflavonoids, the polymethoxylated flavonoids and tocotrienols are preferably present in a ratio of about 75:25 to about 95:5.

In certain preferred embodiments of the present invention the formulations of the present invention inhibit the growth of D-17 cells by 1-99% when measured at 24 hours after administration. In certain further embodiments of the present invention, the growth of D-17 cells is inhibited by at least 10% when measured at 24 hours after administration. In certain further embodiments of the present invention, the growth of D-17 cells is inhibited by at least 20% when measured at 24 hours after administration. In certain further embodiments of the present invention, the growth of D-17 cells is inhibited by at least 30% when measured at 24 hours after administration. In certain further embodiments of the present invention, the growth of D-17 cells is inhibited by at least 40% when measured at 24 hours after administration. In certain further embodiments of the present invention, the growth of D-17 cells is inhibited by at least 50% when measured at 24 hours after administration.

In certain embodiments of the present invention, the formulations of the present invention inhibit the growth of D-17 cells by 1-99% when measured at 48 after administration. In certain further embodiments of the present invention, the growth of D-17 cells is inhibited by at least 10% when measured at 48 hours after administration. In certain embodiments of the present invention, the growth of D-17 cells is inhibited by at least 20% when measured at 48 hours after administration. In certain further embodiments of the present invention, the growth of D-17 cells is inhibited by at least 30% when measured at 48 hours after administration. In certain further embodiments of the present invention, the growth of D-17 cells is inhibited by at least 40% when measured at 48 hours after administration. In certain further embodiments of the present invention, the growth of D-17 cells is inhibited by at least 50% when measured at 48 hours after administration.

In certain embodiments of the present invention, the formulations of the present invention inhibit the growth of D-17 cells by 1-99% when measured at 72 after administration. In certain further embodiments of the present invention, the growth of D-17 cells is inhibited by at least 10% when measured at 72 hours after administration. In certain further embodiments of the present invention, the growth of D-17 cells is inhibited by at least 20% when measured at 72 hours after administration. In certain further embodiments of the present invention, the growth of D-17 cells is inhibited by at least 30% when measured at 72 hours after administration. In certain further embodiments of the present invention, the growth of D-17 cells is inhibited by at least 40% when measured at 72 hours after administration. In certain further embodiments of the present invention, the growth of D-17 cells is inhibited by at least 50% when measured at 72 hours after administration.

In certain embodiments of the present invention, the growth of D-17 cells is inhibited by at least 1%, at least 5%, at least 10%, at least 25%, at least 50%, at least 75%, at least 85%, or at least 95%, when measured at 24, 48, or 72 hours after administration.

In certain embodiments of the present invention, the formulations of the present invention inhibit the growth of MDCK cells by no more than 50% when measured at 24 hours after administration. In certain further embodiments of the present invention, the growth of MDCK cells are inhibited by no more than 40% when measured at 24 hours after administration. In certain further embodiments of the present invention, the growth of MDCK cells are inhibited by no more than 30% when measured at 24 hours after administration. In certain further embodiments of the present invention, the growth of MDCK cells are inhibited by no more than 20% when measured at 24 hours after administration.

In certain embodiments of the present invention, the formulations of the present invention inhibit the growth of MDCK cells by no more than 50% when measured at 48 hours after administration. In certain further embodiments of the present invention, the growth of MDCK cells are inhibited by no more than 40% when measured at 48 hours after administration. In certain further embodiments of the present invention, the growth of MDCK cells are inhibited by no more than 30% when measured at 48 hours after administration. In certain further embodiments of the present invention, the growth of MDCK cells are inhibited by no more than 20% when measured at 48 hours after administration.

In certain embodiments of the present invention, the formulations of the present invention inhibit the growth of MDCK cells by no more than 50% when measured at 72 hours after administration. In certain further embodiments of the present invention, the growth of MDCK cells are inhibited by no more than 40% when measured at 72 hours after administration. In certain further embodiments of the present invention, the growth of MDCK cells are inhibited by no more than 30% when measured at 72 hours after administration. In certain further embodiments of the present invention, the growth of MDCK cells are inhibited by no more than 20% when measured at 72 hours after administration.

In certain embodiments of the present invention, the growth of an MDCK cell is inhibited by no more than 1%, no more than 5%, no more than 10%, no more than 25%, or no more than 50%, when measured at 24, 48 or 72 hours after administration.

### BRIEF DESCRIPTION OF THE FIGURES

FIG. 1 depicts the percent inhibition of cell growth by CPA on MDCK and D-17 cells.

FIGS. 2A and 2B depict the percent control data at 72 h for MDCK and D-17 cells, respectively.

### DETAILED DESCRIPTION

It is postulated that diets rich in whole grains, legumes, fruits and vegetables reduce the risk of various types of cancer and provide beneficial responses due to plant phenolics, largely flavonoids. Flavonoids are a class of chemically related polyphenols of plant origin that are nearly ubiquitous in nature and that also exhibit a broad spectrum of pharmacological properties (Silalahi, 2002; Manthey et al., 2001; Middleton et al., 2000). Of the more-than 4000 naturally occurring flavonoids thus identified, citrus fruit-derived flavonoids and their metabolites have been shown to impart important protective biological action including anticancer and anti-inflammatory activities (Silalahi, 2002; Manthey et al., 2001; Middleton et al., 2000). Polymethoxyflavones (PMFs) are flavonoid compounds having multiple methoxy substituents. Various beneficial effects of flavonoids are described in U.S. Pat. Nos. 6,251,400 and 6,239,114 and in PCT Publication Number WO 01/70029, the disclosures of which are hereby incorporated by reference in their entireties. Other beneficial effects of flavonoid derivatives are discussed in U.S. Pat. Nos. 4,591,600; 5,855,892; and 6,096,364, the disclosures of which are also hereby incorporated by reference in their entireties.

The main PMFs, tangeretin, nobiletin and sinensetin, occur in orange peel. Both tangeretin and nobiletin are present in tangerines sweet orange peel (Citrus sinensis) and in bitter orange peel (Citrus aurantium) (Horowitz and Gentili, 1977). O-PMF (orange peel extract) mixture, such as the one used in the Example of the present application, is rich in PMF such as tangeretin, nobiletin and heptamethoxyflavone. An exemplary list of flavonoids derived from citrus is provided below in Table 1.

TABLE 1
Citrus fruit Bioflavonoids

Grapefruit: apigenin, dihydrokaempferol, eriodictyol,
hesperetin, hesperidin, isorhamnetin,
isosakuranetin, neohesperidin, poncirin,
quercetin, rutin
Lemon: apigenin, apigenin 7-rutinoside,
chrysoeriol, diosmin, eriocitrin,
hesperidin, isorhamnetin, limocitrin
limocitrol, luteolin 7-rutinoside,
naringin, neohesperidin, poncirin,
quercetin
Orange: auranetin, hesperidin, isosakuranetin 7-
rutinoside, naringin, neohesperidin,
nobiletin, rutin, sinensetin, tangeretin,
vitexin
Tangerine: hesperidin, nobiletin, tangeretin

Limonoids occur in significant amounts as aglycones and glycosides in the seeds and fruit of Citrus and species of genera related to Citrus in the plant families Rutaceae and Melinaceae. In vitro and in vivo studies that have examined the anti-neoplastic properties of limonoid aglycones and limonoid glucosides from Citrus have established significant anti-tumor activity among these compounds (Miller et al., 1992; Lam et al., 1994, 2000; Miller et al., 2000; Guthrie et al., 2000) suggesting their importance as natural chemo preventatives in Citrus or as therapeutics of nutraceuticals obtained from Citrus processing co-products.

Citrus fruit tissues and byproducts of juice processing such as peels and molasses are sources of limonoid glucosides and citrus seed contain high concentrations of both limonoid aglycones and glucosides. Limonoid aglycones in the fruit tissues gradually disappear during the late stages of fruit growth and maturation.

Thirty-eight limonoid aglycones have been isolated from citrus. The limonoids are present in three different forms: the dilactone (I) is present as the open D-ring form (monolactone), the limonoate A-ring lactone (II) and the glucoside form (III). Only the monolactones and glucosides are present in fruit tissues. (Hasegawa S. et al., 1994, in Food Phytochemicals for Cancer Prevention I, eds M-T. Huang et al, American Chemical Society, 198-207).

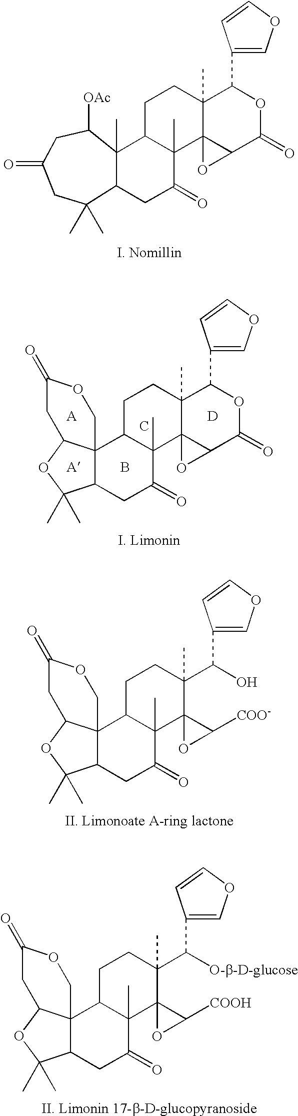

Compound III is the predominant limonoid glucoside found in all juice samples. In orange juice it comprises 56% of the total limonoid glucosides present, while in grapefruit and lemon juices, it comprises an average of 63% to 66% respectively. Procedures for the extraction and isolation of both aglycones and glucosides have been established to obtain concentrated sources of various limonoids (Lam, L. K. T. et al., 1994, in Food Phytochemicals for Cancer Prevention, eds. M. Huang, T. Osawa, C. Ho and R. T. Rosen, ACS Symposium Series 546, p 209).

Canola is a cruciferous crop which is mainly utilized for its extracted oil. After the oil has been extracted a protein rich meal remains which is used as a ruminant in animal diets. Further extraction of the canola meal yields minor components from canola, including, glucosinolates, phenolic acid esters and phenolic acids. The total content of selected minor components in Canola extracts from prior art methods are listed below:

TABLE 2

| | μM/g extract | mg/g extract |
|---|---|---|
| Progoitrin | 8.52 | 3.45 |
| Gluconapin | 5.89 | 2.29 |
| 4-hydroxybrassicin | 3.22 | 1.55 |
| Glucobrassicanapin | 0.90 | 0.36 |
| Glucoalyssin | 0.64 | 0.27 |
| Napoleiferin | 0.54 | 0.23 |
| Glucobrassicin | 0.40 | 0.19 |
| Glucoraphanin | 0.22 | 0.09 |
| Sinigrine | 0.19 | 0.07 |
| Gluconasturtin | 0.19 | 0.08 |
| Neoglucobrassicin | 0.06 | 0.03 |
| 4-methoxyglucobrassicin | traces | — |

Glucosinolates present in the extract from prior art methods from flaked, cooked canola seeds are listed below:

TABLE 3

| | mg/g extract | % content |
|---|---|---|
| Total glucosinolates | 8.61 | 0.9% |
| (flaked, cooked Canola | | |
| seeds) | | |
| Total phenolic acids | 134.00 | 13.4% |
| (flaked, cooked Canola | | |
| seeds) | | |
| Total phenolic acids | 53.15 | 5.3% |
| (Canola meal) | | |
| Free phenolic acids | 246.64 | 24.7% |
| (Canola meal extract after | | |
| hydrolysis) | | |

*The remaining components of extracts are mostly sugars and small amounts and saponins

Content of phenolic acids in the extract from prior art methods from canola meal (mg/g extract) are listed below:

TABLE 4

| | Protocatechuic | Caffeic | p-coumaric | Ferulic | Sinapic |
|---|---|---|---|---|---|
| Free | Trace | 0.03 | 0.02 | 0.02 | 1.03 |
| phenolic | | | | | |
| acids | | | | | |
| Phenolic | Trace | 0.07 | 0.08 | 0.56 | 50.75 |
| acids | | | | | |
| liberated | | | | | |
| from soluble | | | | | |
| esters | | | | | |
| Phenolic | — | Trace | 0.06 | 0.01 | 0.52 |
| acids | | | | | |
| liberated | | | | | |
| from soluble | | | | | |
| glycosides | | | | | |

Content of free phenolic acids in the extract from canola meal after hydrolysis according to prior art methods (ng/g extract) are listed below

TABLE 5

| Protocatechuic | Caffeic | p-coumaric | Ferulic | Sinapic |
|---|---|---|---|---|
| Trace | 0.11 | 0.81 | 3.64 | 242.08 |

Content of phenolic acids in flaked, cooked canola seeds according to prior art methods (mg/g extract) are listed below:

TABLE 6

| | | | p- | | |
|---|---|---|---|---|---|
| | Protocatechuic | Caffeic | coumaric | Ferulic | Sinapic |
| Free | Trace | Trace | Trace | 0.02 | 1.18 |
| phenolic | | | | | |
| acids | | | | | |
| Phenolic acids | Trace | 0.01 | 0.07 | 0.52 | 131.95 |
| liberated from | | | | | |
| soluble esters | | | | | |
| Phenolic acids | — | Trace | Trace | Trace | 0.25 |
| liberated from | | | | | |
| soluble | | | | | |
| glycosides | | | | | |

Preliminary studies suggest that CPA (Canola Phenolic Acid) has antiproliferative and antineoplastic activity both in vitro and in vivo.

Tocotrienols are present in palm oil and are a form of vitamin E having an unsaturated side chain. They include but are not limited to alpha-tocotrienol, gamma-tocotrienol or delta-tocotrienol.

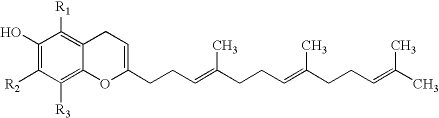

| | R1 | R2 | R3 |
|---|---|---|---|
| α-tocotrienol | CH₃ | CH₃ | CH₃ |
| γ-tocotrienol | H | CH₃ | CH₃ |
| δ-tocotrienol | H | H | CH₃ |

In accordance with certain embodiments of the present invention, flavonoids, limonoids, canola phenolic acid, or combinations thereof may be formulated into pharmaceutical preparations for administration to canines for the treatment of osteosarcoma. In certain further embodiments, the formulations further include one or more tocotrienols.

Many of the flavonoids, limonoids and canola phenolic acid may be provided as compounds with pharmaceutically compatible counterions, a form in which they may be soluble.

Formulations containing the flavonoids, limonoids, canola phenolic acid, tocotrienols, or combinations thereof of the present invention may by administered by any acceptable means including orally, transdermally, rectally, intravenously, intramuscularly, intraperitoneally, subcutaneously, topically, by inhalation or any other means. The oral administration means is preferred.

Formulations suitable for oral administration are commonly known and include liquid solutions of the active compounds dissolved in a diluent such as, for example, saline, water, PEG 400 etc. Solid forms of the compounds for oral administration include capsules or tablets, each comprising the active ingredients and commonly known adjuvants. The active ingredients in the solid dosage form may be present in the form of solids, granules, gelatins, suspensions, and/or emulsions, as will be apparent to persons skilled in the art. The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Formulations suitable for parenteral administration include aqueous and non aqueous isotonic sterile solutions containing buffers, antioxidants, preservatives and any other known adjuvants.

The present invention will now be more fully described with reference to the accompanying example. It should be understood, however, that the following description is illustrative only and should not be taken in any way as a restriction on the generality of the invention specified above.

### EXAMPLE 1

Osteosarcoma is a highly malignant tumor that is the most frequently diagnosed primary bone tumor in dogs. Two canine cell lines MDCK (normal renal epithelial) and D-17 (bone osteosarcoma) were tested in vitro with polymethoxylated flavones (PMF), canola phenolic acid (CPA), orange peel extract (O-PMF) and limonoids. Results indicate that CPA, O-PMF and O-PMF+Limonoid (1:1) may have the potential to inhibit osteosarcoma growth in dogs. The clinical relevance of these in vitro findings remains to be determined, but administration of these products may potentially be indicated as a therapeutic treatment in dogs.

Method

Cell Culture:

PMFs, CPA, O-PMF and Limonoid were tested for their ability to inhibit the growth of canine cells in vitro.

Two canine cell lines were used:
Canine normal renal epithelial cell line MDCK (ATCC CCL-34)
Canine bone osteosarcoma D-17 (ATCC CCL-183)

The cells (10×10³) were cultured in the presence or absence of PMFs, CPA, O-PMF and Limonoids at various concentrations (10-200 μg/ml) for 24, 48 and 72 h at 37° C.

PMF was dissolved in Methanol, O-PMF was dissolved in Saline, CPA and Limonoids were dissolved in DMSO at a concentration of 10000 μg/ml and filter sterilized using 0.2μm syringe filters. The stock solution was diluted to get 800, 200, 100 and 20 μg/ml solutions giving 200-10 μg/ml final concentrations when used in culture. Proliferation of cells was assayed by measuring [³H] thymidine uptake.

Results

Effect of CPA on the Growth of MDCK and D-17 Cells:

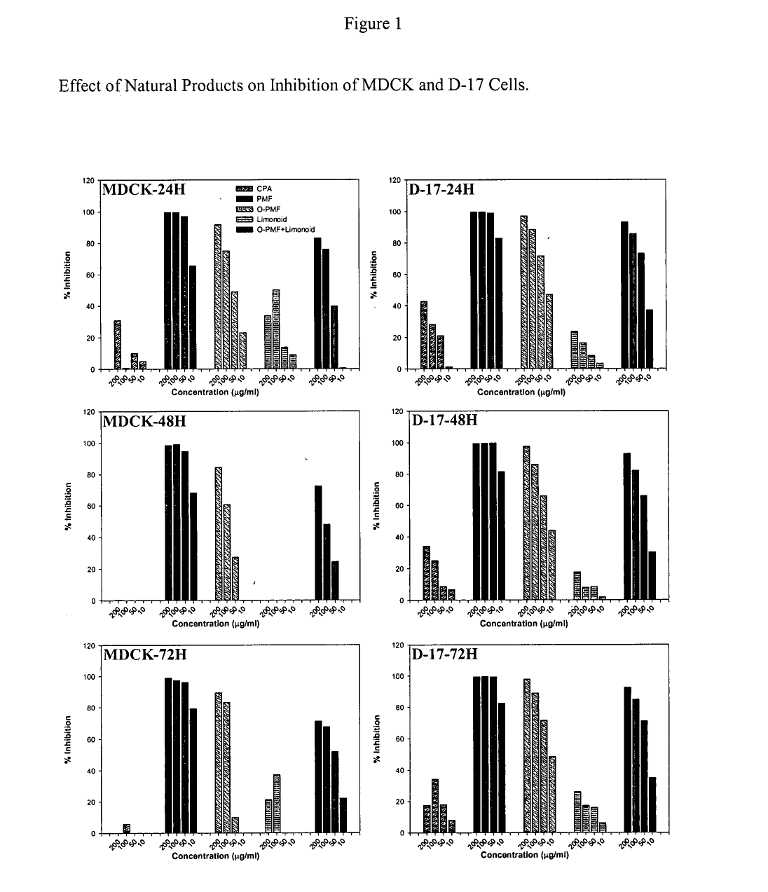

CPA did not have any significant effect on MDCK cell growth. (FIG. 1) Inhibition (31%) was observed only at 24 h at 200 μg/ml and no inhibition was observed after 48 and 72 h of incubation. However, CPA did inhibit the proliferation of D-17 cells in a dose-dependent manner at 24, 48 and 72 h. (FIG. 1) Although the percent inhibition was at a maximum of 40% this effect was consistent in D-17 cells at all time points and doses while the same results were not observed with MDCK cells.

Effect of PMFs on the Growth of MDCK and D-17 Cells:

PMFs inhibited the growth of MDCK cells significantly in a dose dependent manner at all the doses and at all the time points. A similar effect was observed with the D-17 cells (FIG. 1).

Effect of O-PMFs on the Growth of MDCK and D-17 Cells:

O-PMF inhibited the growth of MDCK cells significantly in a dose dependent manner at 24 h. Although O-PMF still inhibited cell proliferation significantly after 48 and 72 h at the dose of 200 μg/ml and 100 μg/ml concentration a significant decrease in inhibitory effect was observed with the other two doses (FIG. 1). No inhibition of growth of MDCK cells was observed with O-PMF at 10 μg/ml after 48 and 72 h incubation while a gradual decrease in percent inhibition was observed with 50 μg/ml. However, O-PMFs inhibited the growth of D-17 cells significantly in a dose dependent manner at all the doses and at all the time points. The percent inhibition was higher than that observed with MDCK cells at all the doses. Also in comparison to the MDCK cells, the percent inhibition of D-17 cells was observed with the lowest concentration up to 72 h of incubation.

Effect of Limonoids on the Growth of MDCK and D-17 Cells:

Limonoids also inhibited the growth of MDCK cells but to a lesser extent. A dose dependent inhibition was observed at 24 h but by 72 h it was only observed at the two highest concentrations (FIG. 1). However, no inhibition of MDCK growth was observed at 48 h time point with the limonoids. On the other hand, Limonoids inhibited the proliferation of D-17 cells in a dose-dependent manner at all time points. Although the percent inhibition was at a maximum of 26% this effect was consistent in D-17 cells and was not observed in MDCK cells.

Effect of O-PMFs and Limonoids on the Growth of MDCK and D-17 Cells:

O-PMF+Limonoids (1:1) also inhibited the growth of MDCK cells. No inhibition of growth occurred at the 10 μg/ml dose at 24 and 48 h but by 72 h there was some growth inhibition of MDCK cells. Comparatively significant inhibition was observed in D-17 cells with O-PMF and Limonoids (1:1) in a dose-dependent manner at all time points (FIG. 1).

IC₅₀ Concentration of PMFs, O-PMFs and O-PMFs+Limonoids for Inhibition of MDCK and D-17 Cell Growth:

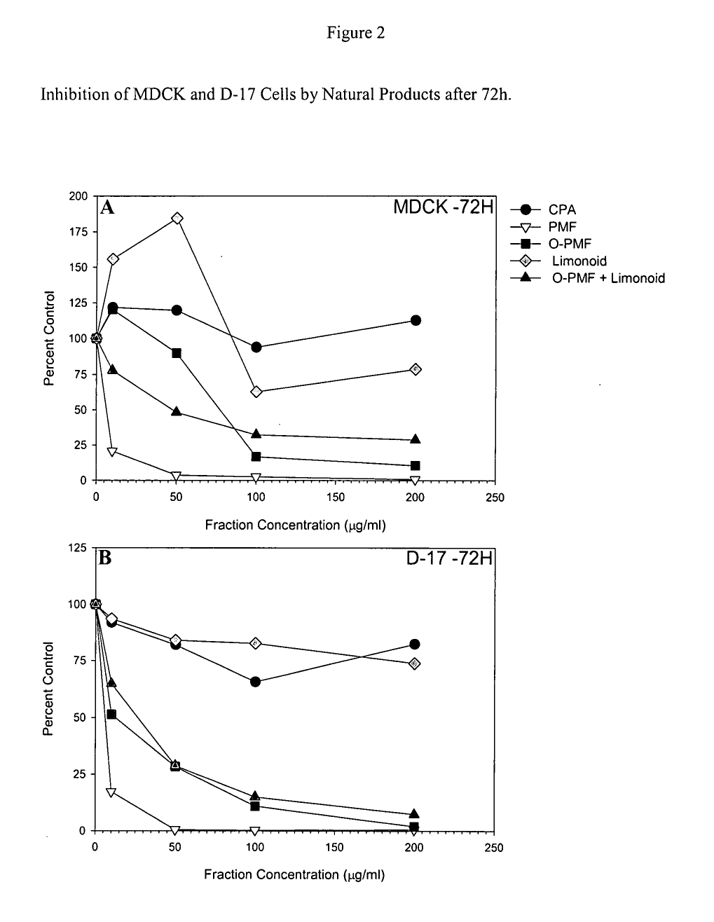

To calculate the IC₅₀ concentration (concentration required to inhibit cell proliferation by 50%) the percent control response of cell proliferation at the different concentrations was calculated. (FIG. 2) The IC₅₀ concentration was then determined and is represented in Table 7. The IC₅₀ concentration for PMFs is similar in both the MDCK and D-17 cells indicating that PMFs are equipotent for both cell types. However the IC₅₀ concentration of O-PMF for MDCK cells (77.5 μg/ml) is higher than D-17 cells (12.5 μg/ml) indicating that O-PMF is more effective in inhibiting the growth of D-17 cells. Similarly the IC₅₀ concentration of O-PMF+Limonoids for MDCK cells (48.5 μg/ml) is higher than D-17 cells (27 μg/ml) indicating that O-PMF+Limonoids is more effective in inhibiting the growth of D-17 cells.

TABLE 7
Effect of PMFs, O-PMFs and O-PMFs and Limonoids on MDCK
and D-17 cells Sensitivity at 72 h.

IC₅₀ (μg/ml)

| Products Tested | MDCK Cells | D-17 Cells |
|---|---|---|
| PMF | 7 | 6 |
| O-PMF | 77.5 | 12.5 |
| O-PMF + Limonoids | 48.5 | 27 |

### CONCLUSION

Results indicate that CPA, O-PMF and O-PMF+Limonoid (1:1) may have the potential to inhibit osteosarcoma growth in dogs. The clinical relevance of these in vitro findings remains to be determined, but administration of these products may potentially be indicated as a therapeutic treatment in dogs.

Many other variations of the present invention will be apparent to those skilled in the art and are meant to be within the scope of the claims appended hereto.

What is claimed:

1. A method of treating canine osteosarcoma comprising administering a composition comprising natural plant based products to a canine in need thereof in an effective amount to inhibit the growth of D-17 cells.

2. The method of claim 1, wherein the natural plant based products are selected from the group consisting of polymethoxyflavones, orange peel extracts, liminoids, canola phenolic acids, and combinations and mixtures thereof.

3. The method of claim 2, wherein the natural plant based product comprises at least one polymethoxyflavone.

4. The method of claim 2, wherein the natural plant based product comprises at least one liminoid.

5. The method of claim 2, wherein the natural plant based product comprises at least one orange peel extract.

6. The method of claim 2, wherein the natural plant based product comprises at least one canola phenolic acid.

7. The method of claim 2, wherein the natural plant based product comprises a mixture of at least one orange peel extract and at least one liminoid.

8. The method of claim 7, wherein the orange peel extract and the liminoid are present in a ratio of 1:1.

9. The method of claim 1, wherein the growth of D-17 cells is inhibited by at least 1%, at least 5%, at least 10%, at least 25%, at least 50%, at least 75%, at least 85%, or at least 95%, when measured at 24 hours after administration.

10. The method of claim 1, wherein the growth of D-17 cells is inhibited by at least 1%, at least 5%, at least 10%, at least 25%, at least 50%, at least 75%, at least 85%, or at least 95%, when measured at 48 hours after administration.

11. The method of claim 1, wherein the growth of D-17 cells is inhibited by at least 1%, at least 5%, at least 10%, at least 25%, at least 50%, at least 75%, at least 85%, or at least 95%, when measured at 72 hours after administration.

12. The method of claim 1, wherein the growth of an MDCK cell is inhibited by no more than 1%, no more than 5%, no more than 10%, no more than 25%, or no more than 50%, when measured at 24 hours after administration.

13. The method of claim 1, wherein the growth of an MDCK cell is inhibited by no more than 1%, no more than 5%, no more than 10%, no more than 25%, or no more than 50%, when measured at 48 hours after administration.

14. The method of claim 1, wherein the growth of an MDCK cell is inhibited by no more than 1%, no more than 5%, no more than 10%, no more than 25%, or no more than 50%, when measured at 72 hours after administration.

15. The method of claim 1, wherein the composition provides an IC₅₀ suitable for the treatment of canine osteosarcoma.
